(19) United States
(12) Patent Application Publication
(10) Pub. No.: US 2010/0136106 A1
(43) Pub. Date: Jun. 3, 2010

(54) Modified Release Famciclovir Compositions

(76) Inventors: Gary Liversidge, Westchester, PA (US); Scott Jenkins, Downington, PA (US); John G. Devane, Barrymore Athione (IE); Paul Stark, Athlone (IE); Niall Fanning, Coosan Athlone (IE); Gurvinder Singh Rekhi, Suwanee, GA (US)

Correspondence Address: Fox Rothschild, LLP;Elan Pharma International Limited, 997 Lenox Drive, Bldg. #3, Lawrenceville, NJ, 08648, US

(21) Appl. No.: 11/568,891

(22) PCT Filed: Jun. 7, 2006
(86) PCT No.: PCT/US06/22120

§ 371 Date: Jan. 15, 2010

Related U.S. Application Data
(60) Provisional application No. 60/688,608, filed on Jun. 8, 2005.

(30) Foreign Application Priority Data
Mar. 10, 2006 (US) 11/372857

(51) Int. Cl. A61K 31/52; A61K 9/48; A61P 31/22; A61P 31/20; A61K 9/58

(52) U.S. Cl. 424/456; 514/263.4; 424/497

(57) ABSTRACT

The invention relates to a multiparticulate modified release composition that, upon administration to a patient, delivers famciclovir in a bimodal, multimodal or continuous manner. The multiparticulate modified release composition comprises a first component and at least one subsequent component, the first component comprising a first population of active ingredient containing particles and the at least one subsequent component comprising a second population of active ingredient containing particles. The invention also relates to a solid oral dosage form containing such a multiparticulate modified release composition, and to a method for the treatment or suppression of viral infections.

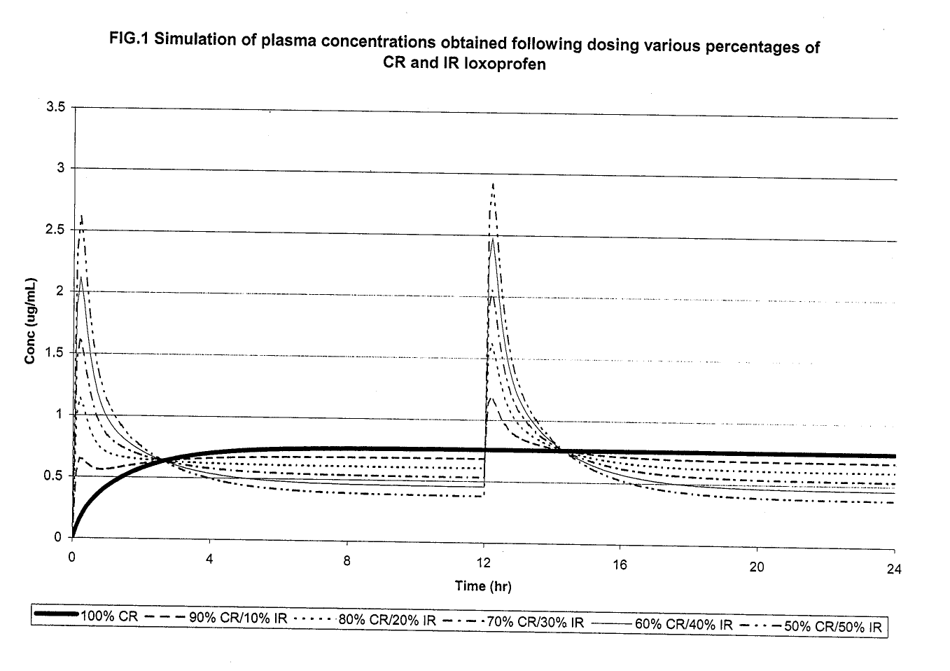

### CROSS-REFERENCE TO RELATED APPLICATIONS

This application claims the benefit of U.S. Provisional Application No. 60/688,608, filed Jun. 7, 2005, and is a continuation-in-part of U.S. application Ser. No. 11/372,857, filed Mar. 10, 2006, which is a continuation-in-part of U.S. application Ser. No. 10/827,689, filed Apr. 19, 2004, which is a continuation of U.S. application Ser. No. 10/354,483, filed Jan. 30, 2003, now U.S. Pat. No. 6,793,936, which in turn is a continuation of U.S. application Ser. No. 10/331,754, filed Dec. 30, 2002, now U.S. Pat. No. 6,902,742, which in turn is a continuation of U.S. application Ser. No. 09/850,425, filed May 7, 2001, now U.S. Pat. No. 6,730,325, which in turn is a continuation of U.S. application Ser. No. 09/566,636, filed May 8, 2000, now U.S. Pat. No. 6,228,398, which in turn is a continuation of PCT Application No. PCT/US99/25632, filed Nov. 1, 1999, which claims the benefit of U.S. Provisional Application No. 60/106,726, filed Nov. 2, 1998, all of which are herein incorporated by reference in their entireties.

### FIELD OF THE INVENTION

The present invention relates to novel compositions and dosage forms for the treatment and suppression of viral infections. In particular, the present invention relates to novel compositions and dosage forms for the delivery of famciclovir and to methods of treatment and suppression using the same.

### BACKGROUND OF THE INVENTION

EP-A-141927 (Beecham Group p.l.c.) discloses penciclovir (PCV), the compound of formula (A):

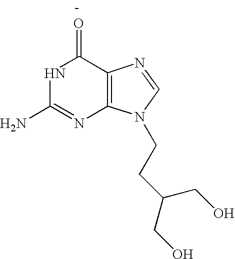

as well as salts, phosphate esters and acyl derivatives thereof. The sodium salt hydrate of penciclovir is disclosed in EP-A-216459 (Beecham Group p.l.c.). Penciclovir is indicated for treatment and suppression of infections caused by, for example, herpes viruses, such as herpes simplex virus type 1 (HSV-1) and herpes simplex virus type 2 (HSV-2), and varicella zoster virus (VZV). Penciclovir and its antiviral activity is also disclosed in Abstract P.V11 5 p. 193 of Abstracts of 14th Int. Congress of Microbiology, Manchester, England 7 13 Sep. 1986 (Boyd et. al.).

Penciclovir can be administered by means of a bioprecursor, or prodrug, which is converted in vivo into penciclovir. Examples of orally active prodrugs of penciclovir are disclosed in EP-A-141927 and EP-A-182024 (Beecham Group p.l.c.). One such prodrug, as described in Example 2 of EP-A-182024, in which the two OH groups are both in the form of acetyl derivatives, is known as famciclovir.

Chemically, famciclovir is known as 2-[2-(2-amino-9H-purin-9-yl)ethyl]-1,3-propanediol diacetate. Its molecular formula is C₁₄H₁₉N₅O₄ and has a molecular weight of 321.2. Famciclovir is a white to pale yellow solid. It is freely soluble in acetone and methanol, and sparingly soluble in ethanol and isopropanol. At 25° C. famciclovir is freely soluble (>25% w/v) in water initially, but rapidly precipitates as the sparingly soluble (2-3% w/v) monohydrate. Famciclovir is not hygroscopic below 85% relative humidity. Partition coefficients are: octanol/water (pH 4.8) P=1.09 and octanol/phosphate buffer (pH 7.4) P=2.08. The preparation of famciclovir is described in Australian patent application No. 84 31,973 and M. R. Hamden et al., J. Med. Chem., vol. 32, 1738 (1989), which are incorporated herein by reference. Famciclovir is currently marketed in the United States under the trademark Famvir® (Novartis Pharmaceuticals Corp. of East Hanover, N.J., U.S.A) in 125 mg, 250 mg, and 500 mg tablets. In such dosage forms, the inactive ingredients include hydroxypropylcellulose, hydroxypropylmethylcellulose, lactose, magnesium stearate, polyethylene glycols, sodium starch glycolate and titanium dioxide.

Famciclovir undergoes rapid biotransformation to the active antiviral compound penciclovir. In cells infected with HSV-1, HSV-2 or VZV, penciclovir is phosphorylated by viral thymidine and cellular kinases to penciclovir triphosphate. Penciclovir triphosphate has been shown to inhibit viral DNA polymerase competitively with deoxyguanine triphosphate, effectively inhibiting viral DNA synthesis and replication.

In addition to its use in the treatment and suppression of herpes and varicella zoster viruses, there is evidence that famciclovir may be effective for use in treatment of the Hepatitis B virus, post therapeutic neuralgia, zoster associated pain and the Epstein Barr virus. See, e.g., U.S. Pat. Nos. 5,246,937; 5,684,153; 5,840,763; 5,866,581; 5,916,893; 5,939,423; 6,093,819; 6,117,857; 6,124,304; 6,288,033; 6,410,055; 6,761,767; and 6,765,007; U.S. Publication No. 2003/0134864; and European Patent No. P182024, each of which is incorporated herein by reference in its entirety.

Famciclovir pharmacokinetics is linear, producing a dose proportional increase in blood plasma levels. Penciclovir concentrations increased in proportion to dose over a famciclovir dose range of 125 mg to 750 mg administered as a single dose. Single oral dose administration of 125 mg, 250 mg or 500 mg famciclovir to healthy male volunteers across 17 studies gave the following pharmacokinetic parameters:

TABLE 2

| | AUC_{(0-∞)} | Cₘₐₓ | Tₘₐₓ |
|---|---|---|---|
| Dose | (mcg · hr/mL) | (mcg/mL) | (h) |
| 125 mg | 2.24 | 0.8 | 0.9 |
| 250 mg | 4.48 | 1.6 | 0.9 |
| 500 mg | 8.95 | 3.3 | 0.9 |

Following single oral-dose administration of 500 mg famciclovir to seven patients with herpes zoster, the mean±SD AUC, Cₘₐₓ, and Tₘₐₓ were 12.1±1.7 mcg·hr/mL, 4.0±0.7 mcg/mL, and 0.7±0.2 hours, respectively. The AUC of penciclovir was approximately 35% greater in patients with herpes zoster as compared to healthy volunteers. Some of this difference may be due to differences in renal function between the two groups.

Famciclovir is absorbed rapidly after oral administration and has a bioavailability of about 69% to about 85%. There is no accumulation of penciclovir after the administration of 500 mg famciclovir t.i.d. for 7 days. When administered at regular doses, food can delay but does not affect the extent of absorption. The time to peak blood levels is about 30 minutes to about 1 hour following a 500 mg oral dose. Penciclovir Cₘₐₓ decreased approximately 50% and Tₘₐₓ was delayed by 1.5 hours when a capsule formulation of famciclovir was administered with food (nutritional content was approximately 910 Kcal and 26% fat). There was no effect on the extent of availability (AUC) of penciclovir. There was an 18% decrease in Cₘₐₓ and a delay in Tₘₐₓ of about 1 hour when famciclovir was given 2 hours after a meal as compared to its administration 2 hours before a meal.

Following oral administration, famciclovir is deacetylated and oxidized to form penciclovir. Metabolites that are inactive include 6-deoxy penciclovir, monoacetylated penciclovir, and 6-deoxy monoacetylated penciclovir (5%, <0.5% and <0.5% of the dose in the urine, respectively). Little or no famciclovir is detected in plasma or urine.

An in vitro study using human liver microsomes demonstrated that cytochrome P450 does not play an important role in famciclovir metabolism. The conversion of 6-deoxy penciclovir to penciclovir is catalyzed by aldehyde oxidase.

Approximately 94% of administered radioactivity was recovered in urine over 24 hours (83% of the dose was excreted in the first 6 hours) after the administration of 5 mg/kg radiolabeled penciclovir as a 1-hour infusion to three healthy male volunteers. Penciclovir accounted for 91% of the radioactivity excreted in the urine.

Following the oral administration of a single 500 mg dose of radiolabeled famciclovir to three healthy male volunteers, 73% and 27% of administered radioactivity were recovered in urine and feces over 72 hours, respectively. Penciclovir accounted for 82% and 6-deoxy penciclovir accounted for 7% of the radioactivity excreted in the urine. Approximately 60% of the administered radiolabeled dose was collected in urine in the first 6 hours.

After intravenous administration of penciclovir in 48 healthy male volunteers, mean±S.D. total plasma clearance of penciclovir was 36.6±6.3 L/hr (0.48±0.09 L/hr/kg). Penciclovir renal clearance accounted for 74.5±8.8% of total plasma clearance. Renal clearance of penciclovir following the oral administration of a single 500 mg dose of famciclovir to 109 healthy male volunteers was 27.7±7.6 L/hr. The plasma elimination half-life of penciclovir was 2.0±0.3 hours after intravenous administration of penciclovir to 48 healthy male volunteers and 2.3±0.4 hours after oral administration of 500 mg famciclovir to 124 healthy male volunteers. The half-life in seven patients with herpes zoster was 3.0±1.1 hours.

The effectiveness of pharmaceutical compounds in the prevention and treatment of disease states depends on a variety of factors including the rate and duration of delivery of the compound from the dosage form to the patient. The combination of delivery rate and duration exhibited by a given dosage form in a patient can be described as its in vivo release profile and, depending on the pharmaceutical compound administered, will be associated with a concentration and duration of the pharmaceutical compound in the blood plasma, referred to as a plasma profile. As pharmaceutical compounds vary in their pharmacokinetic properties such as bioavailability, and rates of absorption and elimination, the release profile and the resultant plasma profile become important elements to consider in designing effective drug therapies.

The release profiles of dosage forms may exhibit different rates and durations of release and may be continuous or pulsatile. Continuous release profiles include release profiles in which one or more pharmaceutical compounds are released continuously, either at a constant or variable rate, and pulsatile release profiles include release profiles in which at least two discrete quantities of one or more pharmaceutical compounds are released at different rates and/or over different time frames. For any given pharmaceutical compound or combination of such compounds, the release profile for a given dosage form gives rise to an associated plasma profile in a patient. Similar to the variables applicable to the release profile, the associated plasma profile in a patient may exhibit constant or variable blood plasma concentration levels of the pharmaceutical compounds in the dosage form over the duration of action and may be continuous or pulsatile. Continuous plasma profiles include plasma profiles of all rates and duration which exhibit a single plasma concentration maximum. Pulsatile plasma profiles include plasma profiles in which at least two higher blood plasma concentration levels of pharmaceutical compound are separated by a lower blood plasma concentration level. Pulsatile plasma profiles exhibiting two peaks may be described as “bimodal.”

When two or more components of a dosage form have different release profiles, the release profile of the dosage form as a whole is a combination of the individual release profiles. The release profile of a two-component dosage form in which each component has a different release profile may described as “bimodal.” For dosage forms of more than two components in which each component has a different release profile, the resultant release profile of the dosage form may be described as “multimodal.” Depending on, at least in part, the pharmacokinetics of the pharmaceutical compounds that are used as well as the specific release profiles of the components of the dosage form, a bimodal or multimodal release profile may result in either a continuous or a pulsatile plasma profile in a patient.

Conventional frequent dosage regimes in which an immediate release (IR) dosage form is administered at periodic intervals typically gives rise to a pulsatile plasma profile. In such cases, a peak in the plasma drug concentration is observed after administration of each IR dose with troughs (regions of low drug concentration) developing between consecutive administration time points. Such dosage regimes (and their resultant pulsatile plasma profiles) can have particular pharmacological and therapeutic effects associated with them that are beneficial for certain drug therapies. For example, the wash out period provided by the fall off of the plasma concentration of the active ingredient between peaks has been thought to be a contributing factor in reducing or preventing patient tolerance to various types of drugs.

Many controlled release drug formulations are aimed at producing a zero order release of the drug compound. Indeed, it is often a specific object of these formulations to minimize the peak to trough variation in plasma concentration levels associated with conventional frequent dosage regimes. For certain drugs, however, some of the therapeutic and pharmacological effects intrinsic in a pulsatile system may be lost or diminished as a result of the constant or nearly constant plasma concentration levels achieved by zero order release drug delivery systems. Thus, modified release compositions or formulations which substantially mimic the release of frequent IR dosage regimes, while reducing the need for frequent dosing, is desirable. Similarly, modified release compositions or formulations which combine the benefits of at least two different release profiles to achieve a resultant plasma profile exhibiting pharmacokinetic values within therapeutically effective parameters is also desirable.

A typical example of a drug which may produce tolerance in patients is methylphenidate. Methylphenidate, or α-phenyl-2-piperidine acetic acid methyl ester, is a stimulant affecting the central nervous and respiratory systems and is primarily used in the treatment of attention deficit hyperactivity disorder (ADHD). After absorption from the gastrointestinal tract (GIT), drug effects persist for 3-6 hours after oral administration of conventional IR tablets or up to about 8 hours after oral administration of extended release formulations. The total dosage is typically in the range of 5-30 mg per day, in exceptional cases rising to 60 mg/day. Under conventional dosage regimes, methylphenidate is given twice daily, typically with one dose given before breakfast and a second dose given before lunch. The last daily dose is preferably given several hours before retiring. Adverse effects associated with methylphenidate treatment include insomnia and the development of patient tolerance.

WO 98/14168 (Alza Corp.) teaches a dosage form and a method of administering methylphenidate in a sustained and constantly ascending rate. The dosage form disclosed comprises a plurality of beads comprising a hydrogel matrix with increasing amounts of the active ingredient therein, coated with varying amounts of a release rate controlling material. Appropriate combinations of the active ingredient dose and the number and thickness coating layers can be selected to give an ascending release profile in which the plasma concentration of the active ingredient continually increases over a given period of time. An object of WO 98/14168 is to release a dosage form at a constantly ascending rate specifically to avoid uneven blood levels (characterized by peaks and troughs) associated with conventional treatments using immediate release dosage formulations. As a result, this formulation does not deliver the active ingredient in either a pulsatile or a bimodal manner.

WO 97/03672 (Chiroscience Ltd.) discloses that methylphenidate exhibits a therapeutic effect when administered in the form of a racemic mixture or in the form of a single isomer (such as the RR d-threo enantiomer). Further, WO 97/03763 (Chiroscience Ltd.) discloses a sustained release formulation containing d-threo methylphenidate (dtmp). This disclosure teaches the use of a composition comprising a coating through which the dtmp passes in order to attain sustained release and achieve serum levels (of the active ingredient) of at least 50% cmax over a period of at least 8 hours. As above, this formulation does not deliver the active ingredient in either a pulsatile or a bimodal manner.

Shah et al., J. Cont. Rel. (1989) 9:169-175 purports to disclose that certain types of hydroxypropyl methylcellulose ethers compressed into a solid dosage form with a therapeutic agent may produce a bimodal release profile. However, it is noted that while polymers from one supplier yielded a bimodal profile, the same polymers with almost identical product specifications obtained from a different source gave non-bimodal release profiles.

Giunchedi et al., Int. J. Pharm (1991) 77:177-181 discloses the use of a hydrophilic matrix multiple-unit formulation for the pulsed release of ketoprofen. Giunchedi et al. teach that ketoprofen is rapidly eliminated from the blood after dosing (plasma half-life 1-3 hours) and consecutive pulses of drug may be more beneficial than constant release for some treatments. The multiple-unit formulation disclosed comprises four identical hydrophilic matrix tablets placed in a gelatin capsule. Although the in vivo studies show two peaks in the plasma profile there is no well defined wash out period and the variation between the peak and trough plasma levels is small.

Conte et al., Drug Dev. Ind. Pharm, (1989) 15:2583-2596 and EP 0 274 734 (Phamidea Srl) teach the use of a three layer tablet for delivery of ibuprofen in consecutive pulses. The three layer tablet is made up of a first layer containing the active ingredient, a barrier layer (the second layer) of semi-permeable material which is interposed between the first layer and a third layer containing an additional amount of active ingredient. The barrier layer and the third layer are housed in an impermeable casing. The first layer dissolves upon contact with a dissolving fluid while the third layer is only available after dissolution or rupture of the barrier layer. In such a tablet the first portion of active ingredient must be released instantly. This approach also requires the provision of a semi-permeable layer between the first and third layers in order to control the relative rates of delivery of the two portions of active ingredient. Additionally, rupture of the semi-permeable layer leads to uncontrolled dumping of the second portion of the active ingredient which may not be desirable.

U.S. Pat. No. 5,158,777 (E. R. Squibb & Sons Inc.) discloses a formulation comprising captopril within an enteric or delayed release coated pH stable core combined with additional captopril which is available for immediate release following administration. In order to form the pH stable core, chelating agents such as disodium edetate or surfactants such as polysorbate 80 are used either alone or in combination with a buffering agent. The compositions have an amount of captopril available for immediate release following oral administration and an additional amount of pH stabilized captopril available for release in the colon.

U.S. Pat. Nos. 4,728,512, 4,794,001 and 4,904,476 (American Home Products Corp.) relate to preparations providing three distinct releases. The preparation contains three groups of spheroids containing an active medicinal substance: the first group of spheroids is uncoated and rapidly disintegrates upon ingestion to release an initial dose of medicinal substance; the second group of spheroids is coated with a pH sensitive coat to provide a second dose; and the third group of spheroids is coated with a pH independent coat to provide to third dose. The preparation is designed to provide repeated release of medicinal substances which are extensively metabolized presystemically or have relatively short elimination half-lives.

U.S. Pat. No. 5,837,284 (Mehta et al) discloses a methylphenidate dosage form having immediate release and delayed release particles. The delayed release is provided by the use of ammonio methacrylate pH independent polymers combined with certain fillers.

Accordingly, it is an object of the present invention to provide a multiparticulate modified release composition comprising at least two populations of active ingredient-containing particles which, upon administration to a patient, exhibits a bimodal or multimodal release profile.

It is another object of the invention to provide a multiparticulate modified release composition comprising at least two populations of active ingredient containing particles which, upon administration to a patient, exhibits a bimodal or multimodal release profile that results in a plasma profile within therapeutically effective pharmacokinetic parameters.

It is a further object of the invention to provide a multiparticulate modified release composition comprising at least two populations of active ingredient containing particles which, upon administration to a patient, exhibits a pulsatile release profile.

It is yet another object of the invention to provide a multiparticulate modified release composition comprising at least two populations of active ingredient containing particles which, upon administration to a patient, results in a pulsatile plasma profile.

It is still another object of the invention to provide a multiparticulate modified release composition comprising at least two populations of active ingredient containing particles which, upon administration to a patient, produces a plasma profile substantially similar to the plasma profile produced by the administration of two or more IR dosage forms given sequentially.

It is yet a further object of the invention to provide a multiparticulate modified release composition comprising at least two populations of active ingredient containing particles which, upon administration to a patient, substantially mimics the pharmacological and therapeutic effects produced by the administration of two or more IR dosage forms given sequentially.

It is still a further object of the invention to provide a multiparticulate modified release composition comprising at least two populations of active ingredient containing particles in which the amount of the one or more active ingredients in the first population of particles is a minor portion of the amount of the one or more active ingredients in the composition, and the amount of the one or more active ingredients in the one or more additional population of particles is a major portion of the amount of the one or more active ingredients in the composition.

It is yet a further object of the invention to provide a solid oral dosage form comprising the multiparticulate modified release composition of the present invention.

Still other objects and advantages of the present invention will become readily apparent to those skilled in the art from the following detailed description, wherein the preferred embodiments of the invention are shown and described, simply by way of illustration of the best mode contemplated of carrying out the invention. As will be realized, the invention is capable of other and different embodiments, and its several details are capable of modifications in various obvious respects, all without departing from the invention.

### SUMMARY OF THE INVENTION

The above objects are realized by a multiparticulate modified release composition having a first component comprising a first population of active ingredient-containing particles and at least a second component comprising a second population of active ingredient-containing particles, wherein each component has a different rate and/or duration of release and wherein at least one of said components comprises famciclovir. The particles of the at least second component are provided in a modified release (MR) form such as, for example, coated with a modified release coating or comprising or incorporated in a modified release matrix material. Upon oral administration to a patient, the composition releases the one or more active ingredients in a bimodal or multimodal manner.

The first component of the multiparticulate modified release composition may exhibit a variety of release profiles including profiles in which substantially all of the active ingredient contained in the first component is released rapidly upon administration of the dosage form, released rapidly but after a time delay (delayed release), or released slowly over time. In one embodiment, the active ingredient contained in the first component of the dosage form is released rapidly upon administration to a patient. As used herein, “released rapidly” includes release profiles in which at least about 80% of the active ingredient of a component of the dosage form is released within about an hour after administration, the term “delayed release” includes release profiles in which the active ingredient of a component of the dosage form is released (rapidly or slowly) after a time delay, and the terms “controlled release” and “extended release” include release profiles in which at least about 80% of the active ingredient contained in a component of the dosage form is released slowly.

The second component of the multiparticulate modified release composition may also exhibit a variety of release profiles including an immediate release profile, a delayed release profile or a controlled release profile. In one embodiment, the second component exhibits a delayed release profile in which the active ingredient of the component is released after a time delay. In another embodiment, the second component exhibits a controlled release profile in which the active ingredient of the component is released over a period of about 12 to about 24 hours after administration.

In two-component embodiments in which the components exhibit different release profiles, the release profile of the active ingredients from the composition is bimodal. In embodiments in which the first component exhibits an immediate release profile and the second component exhibits a delayed release profile, there is a lag time between the release of active ingredient from the first component and the release of the active ingredient from the second component. The duration of the lag time may be varied by altering the amount and/or composition of the modified release coating or by altering the amount and/or composition of the modified release matrix material utilized to achieve the desired release profile. Thus, the duration of the lag time can be designed to mimic a desired plasma profile.

In embodiments in which the first component exhibits an immediate release profile and the second component exhibits a controlled release profile, the active ingredients in the first and second components are released over different time periods. In such embodiments, the immediate release component serves to hasten the onset of action by minimizing the time from administration to a therapeutically effective plasma concentration level, and the one or more subsequent components serve to minimize the variation in plasma concentration levels and/or maintain a therapeutically effective plasma concentration throughout the dosing interval. In one such embodiment, the active ingredient in the first component is released rapidly and the active ingredient in the second component is released within a period of about 12 hours after administration. In another such embodiment, the active ingredient in the first component is released rapidly and the active ingredient in the second component is released within a period of about 24 hours after administration. In yet another such embodiment, the active ingredient in the first component is released rapidly and the active ingredient in the second component is released over a period of about 12 hours after administration. In still another such embodiment, the active ingredient in the first component is released rapidly and the active ingredient in the second component is released over a period of about 24 hours after administration. In yet another such embodiment, the active ingredient in the first component is released rapidly and the active ingredient in the second component is released over a period of at least about 12 hours after administration. In still another such embodiment, the active ingredient in the first component is released rapidly and the active ingredient in the second component is released over a period of at least about 24 hours after administration.

The plasma profile produced by the administration of dosage forms of the present invention which comprise an immediate release component and at least one modified release component can be substantially similar to the plasma profile produced by the administration of two or more IR dosage forms given sequentially, or to the plasma profile produced by the administration of separate IR and MR dosage forms. The modified release composition of the present invention is particularly useful for administering famciclovir which is normally administered two or three times daily. In one embodiment of the present invention, the composition delivers the famciclovir in a bimodal manner. Upon administration, such a composition produces a plasma profile which substantially mimics that obtained by the sequential administration of two IR doses of famciclovir in accordance with a typical treatment regimen. In another embodiment, the composition delivers the famciclovir in a trimodal manner. Upon administration, such a composition produces a plasma profile which substantially mimics that obtained by the sequential administration of three IR doses of famciclovir in accordance with a typical treatment regimen.

According to another aspect of the present invention, the composition can be designed to produce a plasma profile that minimizes or eliminates the variations in plasma concentration levels associated with the administration of two or more IR dosage forms given sequentially. In such embodiments, the composition may be provided with an immediate release component to hasten the onset of action by minimizing the time from administration to a therapeutically effective plasma concentration level, and at least one modified release component to maintain a therapeutically effective plasma concentration level throughout the dosing interval.

The present invention also provides solid oral dosage forms made from the composition of the invention, and for methods for treating an animal, particularly a human, in need of treatment, comprising administering a dosage form comprising a therapeutically effective amount of the composition of the invention to provide bimodal or multimodal release of the active ingredient contained therein.

Advantages of the present invention include reducing the required dosing frequency while still maintaining the benefits derived from a bimodal or multimodal plasma profile. It is also advantageous in terms of patient compliance to have a formulation which may be administered at reduced frequency.

### DETAILED DESCRIPTION OF THE INVENTION

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules, or mixtures thereof, irrespective of their size, shape or morphology.

The term “modified release” as used herein includes a release which is not immediate and includes controlled release, extended release, sustained release and delayed release.

The term “time delay” as used herein refers to the period of time between the administration of a dosage form comprising the composition of the invention and the release of the active ingredient from a particular component thereof.

The term “lag time” as used herein refers to the time between the release of the active ingredient from one component of the composition and the release of the active ingredient from another component of the composition.

The term “erodable” as used herein refers to formulations which may be worn away, diminished, or deteriorated by the action of substances within the body.

The term “diffusion controlled” as used herein refers to formulations which may spread as the result of their spontaneous movement, for example, from a region of higher to one of lower concentration.

The term “osmotic controlled” as used herein refers to formulations which may spread as the result of their movement through a semi-permeable membrane into a solution of higher concentration that tends to equalize the concentrations of the formulation on the two sides of the membrane.

The term “famciclovir” as used herein includes famciclovir, its pharmaceutically acceptable salts, acids, esters, metabolites, complexes or other derivatives and thereof, including penciclovir, and each of their respective stereoisomers including mixtures, racemic or otherwise, of two or more such stereoisomers.

The active ingredients in each component may be the same or different. For example, the composition may comprise components comprising only famciclovir as the active ingredient. Alternatively, the composition may comprise a first component comprising famciclovir and at least one subsequent component comprising an active ingredient other than famciclovir suitable for coadministration therewith, or a first component containing an active ingredient other than famciclovir and at least one subsequent component comprising famciclovir. Indeed, two or more active ingredients may be incorporated into the same component when the active ingredients are compatible with each other. An active ingredient present in one component of the composition may be accompanied by, for example, an enhancer compound or a sensitizer compound in another component of the composition, in order to modify the bioavailability or therapeutic effect thereof.

As used herein, the term “enhancer” refers to a compound which is capable of enhancing the absorption and/or bioavailability of an active ingredient by promoting net transport across the GIT in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

In those embodiments in which more than one a famciclovir-containing component is present, the proportion of famciclovir contained in each component may be the same or different depending on the desired dosing regime. The famciclovir present in the first component and in subsequent components may be any amount sufficient to produce a therapeutically effective plasma concentration level. In one embodiment, the famciclovir is present in the composition in an amount of from about 0.1 to about 500 mg. In another embodiment, the famciclovir is present in the composition in an amount of from about 0.1 to about 250 mg. In yet another embodiment, the famciclovir is present in the composition in an amount of from about 0.1 to about 125 mg.

In embodiments which comprise one or more additional active ingredients, suitable additional active ingredients include any active ingredient for which it is useful to combine the advantages of the release profiles and their associated plasma profiles that are achieved by the compositions of the present invention in order to reduce the dosing frequency may be used in practice of the present invention. Exemplary active ingredients include but are not limited to drug compounds acting on the central nervous system such as psychostimulants and cerebral stimulants, for example methylphenidate; aldosterone inhibitors such as spironolactone, eplerenone and analogs thereof; alkaloids; alpha/beta-blockers such as labetalol, carvedilol and analogs thereof; analgesics such as acetaminophen, tramadol and opioids such as morphine, codeine, thebaine, heroin, oxycodone, hydrocodone, dihydrocodiene, hydromorphone, oxymorphone, buprenorphine, etorphine, naloxone, nicomorphine, methadone, pethidine, fentanyl, alfentanil, sufentanil, remifentanil, carfentanyl, pentazocine, phenazocine, butorphanol, levorphanol and analogs thereof; anesthetics such as lidocaine and bupivacaine and analogs thereof; anorectics such as benzphetamine, diethylproprion, mazindol, phendimetrazine, and phentermine; anti-adrenergic agents such as centrally and peripherally acting anti-adrenergic agents and analogs thereof; anti-allergic agents; anti anginal agents such as nitroglycerine and analogs thereof; anti-arrythmic agents such as moricizine, ibutilide, quinidine, procainamide, disopyramide, lidocaine, tocamide, flecamide, mexiletine, propafenone, bretylium, amiodarone, adenosine, dofetilide and analogs thereof; anti-asthmatic agents such as salbutamol and analogs thereof; antibiotics such as aminosalicylic acid, amoxicillin, amoxicillin and potassium clavulanate, ampicillin, ampicillin and sulbactam, azithromycin, bacampicillin, carbenicillin, carbenicillin indanyl sodium, capreomycin, cefadroxil, cefazolin, cefcapene pivoxil, cephalexin, cephalothin, cephapirin, cephacelor, cefprozil, cephadrine, cefamandole, cefonicide, ceforanide, cefuroxime, cefixime, cefoperazone, cefotaxime, cefpodoxime, ceftaxidime, ceftibuten, ceftizoxime, ceftriaxone, cefepime, cefinetazole, cefotetan, cefoxitin, ciprofloxacine, clarithromycin, clindamycin, clofazimine, cloxacillin, cotriamoxazole, cycloserine, dicloxacillin, dirithromycin, erythromycin, ethambutol, ethionamide, fosfomycin, imipenem, isoniazide, levofloxacine, lomefloxacine, loracarbef, methicillin, methenamine, metronidazole, metoclopramide, mezlocillin, nafcillin, nalidixic acid, nitrofurantoin, norfloxacin, novobiocin, ofloxacin, oxacillin, penicillin, pentamidine, piperacillin, piperacillin and tazobactam, sparfloxacin, sulphacytine, sulphamerazine, sulphamethazine, sulphamethixole, sulphasalazine, sulphisoxazole, sulphapyrizine, sulphadiazine, sulphmethoxazole, sulphapyridine, ticarcillin, ticarcillin and potassium clavulanate, trimethoprime, trimetrexate, troleanomycin, vancomycin, verapamil and analogs thereof; anti-cancer agents; anti coagulant agents such as heparin, hirudin and analogs thereof; anti-convulsants such as carbamazepine, levetiracetam, topiramate and analogs thereof; anti-depressant agents such as amitriptyline, amoxapine, bupropion, citalopram, clomipramine, desipramine, doxepin, escitalopram, fluoxetine, fluvoxamine, imipramine, maprotiline, mirtazapine, nefazodone, nortriptyline, paroxetine, phenelzine, protriptyline, sertraline, tranylcypromine, trazodone, trimipramine, venlafaxine, and analogs thereof; anti-diabetic agents; anti-diarrheal agents such as loperamide and analogs thereof; anti emetic agents such as scopolamine, ondansetron, domperidone, metoclopramide and analogs thereof; anti-epileptic agents; anti-fungal agents such as acylanilide and analogs thereof; antihistamines such as terfenadine and analogs thereof; anti-hypertensive agents; anti-inflammatory agents; anti migraine agents such as sumatriptan, ergot alkaloids and analogs thereof; anti neoplastics such as fluorouracil, bleomycin and analogs thereof; anti-parkinsonian agents other than carbidopa, levodopa or entacapone; anti-psychotic agents such as acetophenazine, aripiprazole, chlorprothixene, droperidol, olanzapine, promazine, quetiapine, risperidone, sulpiride, triflupromazine, ziprasidone, and analogs thereof; anti-rheumatic agents such as fentiazac and analogs thereof; anti-thrombic agents; anti-tussive agents; anti-ulcer agents such as 5-asa, cimetidine, famotidine, lansoprazole, omeprazole, ranitidine and analogs thereof; anti-viral agents such as acyclovir, ganciclovir, zidovudine and analogs thereof; anxiolytic agents such as alprazolam, buspirone, clonazepam, clorazepate, chlordiazepoxide, diazepam, hydroxyzine, lorazepam, meprobamate, oxazepam, and analogs thereof; ARB blockers, such as irbesartan, candesartan, losartan, valsartan, telmisartan, eprosartan and analogs thereof; beta-blockers, such as acebutolol, atenolol, betaxolol, bisoprolol, esmolol, metoprolol, carteolol, nadolol, penbutolol, pindolol, propanolol, sotalol, timolol, labetalol and analogs thereof; blood lipid-lowering agents such statins such as simvastatin and analogs thereof; calcium channel blockers such as nifedipine, verapamil, diltiazem, nicardipine, nisoldipine, nimodipine, isradipine, bepridil, felodipine, amlodipine and analogs thereof; cardiovascular agents, anti hypertensive agents and vasodilators such as benazepril, captopril, clonidine, enelapril, fosinopril, isosorbide dinitrate, isosorbide 5 mononitrate, hydralizine, lisinopril, moexipril, pentoxifylline, perindopril, prazosine, quinapril, quinidine, ramipril, trandolapril, nitrates, peripheral vasodilators and analogs thereof; chelating agents such as deferoxamine and analogs thereof; chemotherapy agents such as vincristine and analogs thereof; contraceptives; diuretic agents such as loop diuretics, acetazolamide, amiloride, bendroflumethiazide, bumetanide, chlorthalidone, chlorothiazide, dichlorphenamide, ethacrynic acid, furoseamide, hydrochlorothiazide, hydroflumethiazide, indapamide, mannitol, methazolamide, methyclothiazide, metolazone, naturetin, polythiazide, spironolactone, triameterene, triamterene, trichlormethiazide, triamterene, torsemide, and analogs thereof; fertility promoters; hypnotic agents such as amobarbital, butabarbital, chloral hydrate, estazolam, flurazepam, mephobarbital, paraldehyde, pentobarbital, phenobarbital, quazepam, secobarbital, temazepam, triazolam, zaleplon, zolpidem and analogs thereof; inducers and inhibitors of uterine labor; inotropic agents such as digoxin and analogs thereof; narcotic antagonists; NSAIDs such as celecoxib, etoricoxib, rofecoxib, valdecoxib, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, tiaprofenic acid, salicylates such as acetylsalicylic acid, choline magnesium salicylate, choline salicylate, magnesium salicylate, and sodium salicylate, and analogs thereof; neuroleptic agents; synthetic and naturally occurring peptides, proteins or hormones such as desmopressin, vasopressin, insulin, calcitonin, calcitonin gene regulating protein, atrial natriuretic protein, colony stimulating factor, betaseron, erythropoietin (EPO), interferons such as α, β or γ interferon, somatropin, somatotropin, somastostatin, insulin like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin 2 and analogs thereof; prostaglandins and analogs thereof; sedatives such as benzodiazepines, phenothiozines and analogs thereof; and vasoprotective agents.

It will be understood that suitable additional active ingredients also include all pharmaceutically acceptable salts, acids, esters, complexes or other derivatives of the active ingredients recited above, and may be present either in the form of one stereoisomer or as a mixture, racemic or otherwise, of stereoisomers.

The time release characteristics for the delivery of famciclovir from each of the components may be varied by modifying the composition of each component, including modifying any of the excipients and/or coatings which may be present. In particular, the release of famciclovir may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired, delay time for each particular component. The modified release coating may be preset, in each component, in any amount that is sufficient to yield the desired time lag between components.

The lag time and/or time delay for the release of famciclovir from each famciclovir-containing component may also be varied by modifying the composition of each of the components, including modifying any excipients and coatings which may be present. For example, the first component may be an immediate release component wherein the famciclovir is released immediately upon administration. Alternatively, the first component may be, for example, a time-delayed immediate release component in which famciclovir is released substantially in its entirety immediately after a time delay. The second and subsequent component may be, for example, a time-delayed immediate release component as just described or, alternatively, a time-delayed sustained release or extended release component in which famciclovir is released in a controlled fashion over an extended period of time.

As will be appreciated by those skilled in the art, the exact nature of the plasma concentration curve will be influenced by the combination of all of these factors just described. In particular, the lag time between the delivery (and thus also the onset of action) of famciclovir in each famciclovir-containing component may be controlled by varying the composition and coating (if present) of each of the components. Thus by variation of the composition of each component (including the amount and nature of the active ingredient(s)) and by variation of the lag time, numerous release and plasma profiles may be obtained. Depending on the duration of the lag time between the release of famciclovir from each such component and the nature of the release of famciclovir from such each component (i.e. immediate release, sustained release etc.), the plasma profile may be continuous (i.e., having a single maximum) or pulsatile in which the peaks in the plasma profile may be well separated and clearly defined (e.g. when the lag time is long) or superimposed to a degree (e.g. when the lag time is short).

The plasma profile produced from the administration of a single dosage unit comprising the composition of the present invention is advantageous when it is desirable to deliver two or more pulses of active ingredient without the need for administration of two or more dosage units. Additionally, in the case of treating viral infections, it is particularly useful to have such a multimodal plasma profile. For example, typical famciclovir treatment regimes consists of the administration of either two doses of an immediate release dosage formulation given twelve hours apart or three doses of an immediate release dosage formulation given eight hours apart for a period of seven days. These types of regimes have been found to be therapeutically effective and are widely used.

Any coating material which modifies the release of famciclovir in the desired manner may be used. In particular, coating materials suitable for use in the practice of the present invention include but are not limited to polymer coating materials such as cellulose acetate phthalate, cellulose acetate trimaletate, hydroxypropylmethylcellulose phthalate, polyvinyl acetate phthalate, ammoniomethacrylate copolymers such as those sold under the trademark Eudragit® RS and RL, poly (acrylic acid) and polyacrylate and methacrylate copolymers such as those sold under the trademark Eudragit® S and L, polyvinyl acetaldiethylamino acetate, hydroxypropylmethylcellulose acetate succinate, shellac, hydrogels and gel-forming materials such as carboxyvinyl polymers, sodium alginate, sodium carmellose, calcium carmellose, sodium carboxymethyl starch, polyvinyl alcohol, hydroxyethylcellulose, methylcellulose, gelatin, starch, and cellulose based cross-linked polymers—in which the degree of crosslinking is low so as to facilitate adsorption of water and expansion of the polymer matrix, hydroxypropylcellulose, hydroxypropyl-methylcellulose, polyvinylpyrrolidone, crosslinked starch, microcrystalline cellulose, chitin, aminoacrylmethacrylate copolymer (Eudragit® RS-PM, Rohm & Haas), pullulan, collagen, casein, gum arabic, sodium carboxymethylcellulose, (swellable hydrophilic polymers) poly(hydroxyalkyl methacrylate) (mol. wt. ˜5 k-5,000 k), polyvinylpyrrolidone (mol. wt. ˜10 k-360 k), anionic and cationic hydrogels, polyvinyl alcohol having a low acetate residual, a swellable mixture of agar and carboxymethylcellulose, copolymers of maleic anhydride and styrene, ethylene, propylene or isobutylene, pectin (mol. wt. ˜30 k-300 k), polysaccharides such as agar, acacia, karaya, tragacanth, algins and guar, polyacrylamides, Polyox® polyethylene oxides (mol. wt. ˜100 k-5,000 k), AquaKeep® acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methylcellulose, sodium or calcium carboxymethylcellulose, hydroxypropylmethylcellulose, hydroxypropylcellulose, hydroxyethylcellulose, nitrocellulose, carboxymethylcellulose, cellulose ethers, polyethylene oxides (e.g. Polyox®, Union Carbide), methylethylcellulose, ethylhydroxyethylcellulose, cellulose acetate, cellulose butyrate, cellulose propionate, gelatin, collagen, starch, maltodextrin, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of famciclovir dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixture thereof.

A modified release composition according to the present invention may be incorporated into any suitable dosage form which facilitates release of the active ingredient in a pulsatile manner. In one embodiment, the dosage form comprises a blend of different populations of active ingredient-containing particles which make up the immediate release and the modified release components, the blend being filled into suitable capsules, such as hard or soft gelatin capsules. Alternatively, the different individual populations of active ingredient-containing particles may be compressed (optionally with additional excipients) into mini-tablets which may be subsequently filled into capsules in the appropriate proportions. Another suitable dosage form is that of a multilayer tablet. In this instance the first component of the modified release composition may be compressed into one layer, with the second component being subsequently added as a second layer of the multilayer tablet. The populations of famciclovir-containing particles making up the composition of the invention may further be included in rapidly dissolving dosage forms such as an effervescent dosage form or a fast-melt dosage form.

In one embodiment, the composition and the solid oral dosage forms containing the composition release famciclovir such that substantially all of the famciclovir contained in the first component is released prior to release of famciclovir from the at least one second component. When the first component comprises an IR component, for example, it is preferable that release of famciclovir from the at least one second component is delayed until substantially all famciclovir in the IR component has been released. Release of famciclovir from the at least one second component may be delayed as detailed above by the use of a modified release coatings and/or a modified release matrix material.

When it is desirable to minimize patient tolerance by providing a dosage regime which facilitates wash-out of a first dose of famciclovir from a patient's system, release of famciclovir from subsequent components may be delayed until substantially all of the famciclovir contained in the first component has been released, and further delayed until at least a portion the famciclovir released from the first component has been cleared from the patient's system. In one embodiment, the release of famciclovir from subsequent components of the composition is substantially, if not completely, delayed for a period of at least about six hours after administration of the composition. In another embodiment, the release of famciclovir from subsequent components of the composition is substantially, if not completely, delayed for a period of at least about twelve hours after administration of the composition.

As described hereinbelow, the present invention also includes various types of modified release systems by which famciclovir may be delivered in either a pulsatile or continuous manner. These systems include but are not limited to: films with famciclovir in a polymer matrix (monolithic devices); famciclovir contained by the polymer (reservoir devices); polymeric colloidal particles or microencapsulates (microparticles, microspheres or nanoparticles) in the form of reservoir and matrix devices; famciclovir contained by a polymer containing a hydrophilic and/or leachable additive e.g., a second polymer, surfactant or plasticizer, etc. to give a porous device, or a device in which the release of famciclovir may be osmotically controlled (both reservoir and matrix devices); enteric coatings (ionizable and dissolve at a suitable pH); (soluble) polymers with (covalently) attached pendant drug molecules; and devices where release rate is controlled dynamically: e.g., the osmotic pump.

The delivery mechanism of the present invention can control the rate of release of famciclovir. While some mechanisms will release famciclovir at a constant rate, others will vary as a function of time depending on factors such as changing concentration gradients or additive leaching leading to porosity, etc.

Polymers used in sustained release coatings are necessarily biocompatible, and ideally biodegradable. Examples of both naturally occurring polymers such as Aquacoat® (FMC Corporation, Food & Pharmaceutical Products Division, Philadelphia, USA) (ethylcellulose mechanically spheronised to sub-micron sized, aqueous based, pseudo-latex dispersions), and also synthetic polymers such as the Eudragit® (Röhm Pharma, Weiterstadt) range of poly(acrylate, methacrylate) copolymers are known in the art.

In one approach, a modified release is achieved by encapsulation or containment of the drug entirely (e.g., as a core) within a polymer film or coat (i.e., microcapsules or spray/pan coated cores). The various factors that can affect the diffusion process may readily be applied to reservoir devices (e.g., the effects of additives, polymer functionality (and, hence, sink-solution pH) porosity, film casting conditions, etc.) and, hence, the choice of polymer must be an important consideration in the development of reservoir devices. Modeling the release characteristics of reservoir devices and monolithic devices in which the transport of the drug is by a solution-diffusion mechanism therefore typically involves a solution to Fick's second law (unsteady-state conditions; concentration dependent flux) for the relevant boundary conditions. When the device contains dissolved active agent, the rate of release decreases exponentially with time as the concentration (activity) of the agent (i.e., the driving force for release) within the device decreases (i.e., first order release). If, however, the active agent is in a saturated suspension, then the driving force for release is kept constant until the device is no longer saturated. Alternatively the release-rate kinetics may be desorption controlled, and a function of the square root of time.

Transport properties of coated tablets, may be enhanced compared to free-polymer films, due to the enclosed nature of the tablet core (the permeant) which may enable the internal build-up of an osmotic pressure which will then act to force the permeant out of the tablet.

The effect of de-ionized water on salt containing tablets coated in poly(ethylene glycol) (PEG)-containing silicone elastomer, and also the effects of water on free films has been investigated. The release of salt from the tablets was found to be a mixture of diffusion through water filled pores, formed by hydration of the coating, and osmotic pumping. KCl transport through films containing just 10% PEG was negligible, despite extensive swelling observed in similar free films, indicating that porosity was necessary for the release of the KCl which then occurred by trans-pore diffusion. Coated salt tablets, shaped as disks, were found to swell in de-ionized water and change shape to an oblate spheroid as a result of the build-up of internal hydrostatic pressure: the change in shape providing a means to measure the force generated. As might be expected, the osmotic force decreased with increasing levels of PEG content. The lower PEG levels allowed water to be imbibed through the hydrated polymer, while the porosity resulting from the coating dissolving at higher levels of PEG content (20 to 40%) allow the pressure to be relieved by the flow of KCl.

Methods and equations have been developed, which by monitoring (independently) the release of two different salts (e.g., KCl and NaCl) allowed the calculation of the relative magnitudes that both osmotic pumping and trans-pore diffusion contributed to the release of salt from the tablet. At low PEG levels, osmotic flow was increased to a greater extent than was trans-pore diffusion due to the generation of only a low pore number density: at a loading of 20%, both mechanisms contributed approximately equally to the release. The build-up of hydrostatic pressure, however, decreased the osmotic inflow, and osmotic pumping. At higher loadings of PEG, the hydrated film was more porous and less resistant to outflow of salt. Hence, although the osmotic pumping increased (compared to the lower loading), trans-pore diffusion was the dominant release mechanism. An osmotic release mechanism has also been reported for microcapsules containing a water soluble core.

Monolithic (matrix) devices, where the active agent is provided within a polymer matrix, are commonly used for controlling the release of drugs. Such devices are typically formed by the compression of a polymer/drug mixture or by dissolution or melting. In contrast to reservoir devices, the danger of an accidental high dosage that could result from the rupture of the membrane of a reservoir device is not present in the monolithic device.

The release properties of monolithic devices may be dependent upon a variety of factors including whether the drug is dispersed or dissolved in the polymer, the solubility of the drug in the polymer matrix and, in the case of porous matrices, the solubility in the sink solution within the particle's pore network and the tortuosity of the network. For low loadings of drug, (0 to 5% w/v) the drug will be released by a solution-diffusion mechanism (in the absence of pores). At higher loadings (5 to 10% w/v), the release mechanism will be complicated by the presence of cavities formed near the surface of the device as the drug is lost: such cavities fill with fluid from the environment increasing the rate of release of the drug.

It is common to add a plasticizer (e.g., a poly(ethylene glycol)), a surfactant, or adjuvant (i.e., an ingredient which increases effectiveness), to monolithic devices and reservoir devices as a means to enhance the permeability (although, in contrast, plasticizers may be fugitive, and simply serve to aid film formation and, hence, decrease permeability—a property normally more desirable in polymer paint coatings). It has been noted that the leaching of PEG increased the permeability of (ethyl cellulose) films linearly as a function of PEG loading by increasing the porosity, however, the films retained their barrier properties, not permitting the transport of electrolyte. It was deduced that the enhancement of their permeability was as a result of the effective decrease in thickness caused by the PEG leaching. This was evidenced from plots of the cumulative permeant flux per unit area as a function of time and film reciprocal thickness at a PEG loading of 50% w/w: plots showing a linear relationship between the rate of permeation and reciprocal film thickness, as expected for a (Fickian) solution-diffusion type transport mechanism in a homogeneous membrane. Extrapolation of the linear regions of the graphs to the time axis gave positive intercepts on the time axis: the magnitude of which decreased towards zero with decreasing film thickness. These changing lag times were attributed to the occurrence of two diffusional flows during the early stages of the experiment (the flow of the drug and also the flow of the PEG), and also to the more usual lag time during which the concentration of permeant in the film is building-up. Caffeine, when used as a permeant, showed negative lag times. No explanation of this was forthcoming, but it was noted that caffeine exhibited a low partition coefficient in the system, and that this was also a feature of aniline permeation through polyethylene films which showed a similar negative time lag.

The effects of added surfactants on hydrophobic matrix devices has been investigated. It was thought that surfactant may increase the drug release rate by three possible mechanisms: (i) increased solubilization, (ii) improved “wettability” to the dissolution media, and (iii) pore formation as a result of surfactant leaching. For the system studied (Eudragit® RL 100 and RS 100 plasticized by sorbitol, flurbiprofen as the drug, and a range of surfactants) it was concluded that improved wetting of the tablet led to only a partial improvement in drug release (implying that the release was diffusion, rather than dissolution, controlled), although the effect was greater for Eudragit® RS than Eudragit® RL, while the greatest influence on release was by those surfactants that were more soluble due to the formation of disruptions in the matrix allowing the dissolution medium access to within the matrix. This is of obvious relevance to a study of latex films which might be suitable for pharmaceutical coatings, due to the ease with which a polymer latex may be prepared with surfactant as opposed to surfactant-free. Differences were found between the two polymers with only the Eudragit® RS showing interactions between the anionic/cationic surfactant and drug. This was ascribed to the differing levels of quaternary ammonium ions on the polymer.

Composite devices consisting of a polymer/drug matrix coated in a polymer containing no drug also exist. Such a device was constructed from aqueous Eudragit® lattices, and was found to provide a continuous release by diffusion of the drug from the core through the shell. Similarly, a polymer core containing the drug has been produced and coated with a shell that was eroded by gastric fluid. The rate of release of the drug was found to be relatively linear (a function of the rate limiting diffusion process through the shell) and inversely proportional to the shell thickness, whereas the release from the core alone was found to decrease with time.

Methods for the preparation of hollow microspheres have been described. Hollow microspheres were formed by preparing a solution of ethanol/dichloromethane containing the drug and polymer. On pouring into water, an emulsion is formed containing the dispersed polymer/drug/solvent particles, by a coacervation-type process from which the ethanol rapidly diffused precipitating polymer at the surface of the droplet to give a hard-shelled particle enclosing the drug dissolved in the dichloromethane. A gas phase of dichloromethane was then generated within the particle which, after diffusing through the shell, was observed to bubble to the surface of the aqueous phase. The hollow sphere, at reduced pressure, then filled with water which could be removed by a period of drying. No drug was found in the water. Highly porous matrix-type microspheres have also been described. The matrix-type microspheres were prepared by dissolving the drug and polymer in ethanol. On addition to water, the ethanol diffused from the emulsion droplets to leave a highly porous particle. A suggested use of the microspheres was as floating drug delivery devices for use in the stomach.

Pendent devices for attaching a range of drugs such, as for example, analgesics and antidepressants, etc., by means of an ester linkage to poly(acrylate) ester latex particles prepared by aqueous emulsion polymerization has been developed. These lattices, when passed through an ion exchange resin such that the polymer end groups were converted to their strong acid form, could self-catalyze the release of the drug by hydrolysis of the ester link.

Drugs have been attached to polymers, and also monomers have been synthesized with a pendent drug attached. Dosage forms have been prepared in which the drug is bound to a biocompatible polymer by a labile chemical bond e.g., polyanhydrides prepared from a substituted anhydride (itself prepared by reacting an acid chloride with the drug: methacryloyl chloride and the sodium salt of methoxy benzoic acid) were used to form a matrix with a second polymer (Eudragit® RL) which released the drug on hydrolysis in gastric fluid. The use of polymeric Schiff bases suitable for use as carriers of pharmaceutical amines has also been described.

Enteric coatings and films consist of pH sensitive polymers. Typically the polymers are carboxylated and interact very little with water at low pH, while at high pH the polymers ionize causing swelling or dissolving of the polymer. Coatings and films can therefore be designed to remain intact in the acidic environment of the stomach, protecting either the drug from this environment or the stomach from the drug, but to dissolve in the more alkaline environment of the intestine.

Osmotically controlled devices such as an osmotic pump are similar to a reservoir device but contain an osmotic agent (e.g., the active agent in salt form) which acts to imbibe water from the surrounding medium via a semi-permeable membrane. Such a device, called an elementary osmotic pump, has been described. Pressure is generated within the device which forces the active agent out of the device via an orifice of a size designed to minimize solute diffusion, while preventing the build-up of a hydrostatic pressure head which can have the effect of decreasing the osmotic pressure and changing the dimensions of the device. While the internal volume of the device remains constant, and there is an excess of solid or saturated solution in the device, then the release rate remains constant delivering a volume equal to the volume of solvent uptake.

Monolithic devices have been prepared using polyelectrolyte gels which swell when, for example, an external electrical stimulus is applied causing a change in pH. The release may be modulated by changes in the applied current to produce a constant or pulsatile release profile.

In addition to their use in drug matrices, hydrogels find use in a number of biomedical applications such as, for example, soft contact lenses, and various soft implants, and the like.

According to another aspect of the present invention, there is provided a method for treating a patient suffering from viral infection, such as HSV-1, HSV-2 or VZV, comprising the step of administering a therapeutically effective amount of the composition of the present invention in solid oral dosage form. Advantages of the method of the present invention include a reduction in the dosing frequency required by conventional multiple IR dosage regimes while still maintaining the benefits derived from a pulsatile plasma profile or eliminating or minimizing the variations in plasma concentration levels. This reduced dosing frequency is advantageous in terms of patient compliance and the reduction in dosage frequency made possible by the method of the present invention would contribute to controlling health care costs by reducing the amount of time spent by health care workers on the administration of drugs.

In the following examples, all percentages are weight by weight unless otherwise stated. The term “purified water” as used throughout the Examples refers to water that has been purified by passing it through a water filtration system. It is to be understood that the examples are for illustrative purposes only, and should not be interpreted as restricting the spirit and breadth of the invention as defined by the scope of the claims that follow.

### Example 1

A multiparticulate modified release composition according to the present invention comprising an immediate release component and a modified release component each containing famciclovir is prepared as follows.

### (a) Immediate Release Component

A solution of famciclovir is prepared according to any of the formulations given in Table 1. The methylphenidate solution is then coated onto nonpareil seeds to a level of approximately 16.9% solids weight gain using, for example, a Glatt GPCG3 (Glatt, Protech Ltd., Leicester, UK) fluid bed coating apparatus to form the IR particles of the immediate release component.

TABLE 1
Immediate release component solutions

| | Amount (% (w/w)) | Amount (% (w/w)) |
|---|---|---|
| Ingredient | (i) | (ii) |
| Famciclovir | 13.0 | 13.0 |
| Polyethylene Glycol 6000 | 0.5 | 0.5 |
| Polyvinylpyrrolidone | 3.5 | |
| Purified Water | 83.5 | 86.5 |

### (b) Modified Release Component

Delayed release particles containing famciclovir are prepared by coating immediate release particles prepared according to Example 1(a) above with a modified release coating solution as detailed in Table 2. The immediate release particles are coated to varying levels up to approximately to 30% weight gain using, for example, a fluid bed apparatus.

TABLE 2
Modified release component coating solutions

| | Amount, % (w/w) | | | | | | | |
|---|---|---|---|---|---|---|---|---|
| Ingredient | (i) | (ii) | (iii) | (iv) | (v) | (vi) | (vii) | (viii) |
| Eudragit ® | 49.7 | 42.0 | 47.1 | 53.2 | 40.6 | — | — | 25.0 |
| RS 12.5 | | | | | | | | |
| Eudragit ® | — | — | — | — | — | 54.35 | 46.5 | — |
| S 12.5 | | | | | | | | |
| Eudragit ® | — | — | — | — | — | — | 25.0 | — |
| L 12.5 | | | | | | | | |
| Polyvinyl- | — | — | — | 0.35 | 0.3 | — | — | — |
| pyrrolidone | | | | | | | | |
| Diethyl- | 0.5 | 0.5 | 0.6 | 1.35 | 0.6 | 1.3 | 1.1 | — |
| phthalate | | | | | | | | |
| Triethyl | — | — | — | — | — | — | — | 1.25 |
| citrate | | | | | | | | |
| Isopropyl | 39.8 | 33.1 | 37.2 | 45.1 | 33.8 | 44.35 | 49.6 | 46.5 |
| alcohol | | | | | | | | |
| Acetone | 10.0 | 8.3 | 9.3 | — | 8.4 | — | — | — |
| Talc¹ | — | 16.0 | 5.9 | — | 16.3 | — | 2.8 | 2.25 |

¹Talc is simultaneously applied during coating for formulations in column (i), (iv) and (vi).

### (c) Encapsulation of Immediate and Delayed Release Particles.

The immediate and delayed release particles prepared according to Example 1(a) and (b) above are encapsulated in size 2 hard gelatin capsules to an overall 20 mg dosage strength using, for example, a Bosch GKF 4000S encapsulation apparatus. The overall dosage strength of 20 mg of famciclovir was made up of 10 mg from the immediate release component and 10 mg from the modified release component.

### Example 2

A multiparticulate modified release composition according to the present invention comprising an immediate release component and a modified release component comprising a modified release matrix material is prepared according to the formulations shown in Table 3(a) and (b).

TABLE 3 (a)
100 mg of IR component is encapsulated with 100 mg of modified
release (MR) component to give a 20 mg dosage strength product

| | % (w/w) |
|---|---|
| IR component: | |
| Famciclovir | 10 |
| Microcrystalline cellulose | 40 |
| Lactose | 45 |
| Povidone | 5 |
| MR component | |
| Famciclovir | 10 |
| Microcrystalline cellulose | 40 |
| Eudragit ® RS | 45 |
| Povidone | 5 |

TABLE 3 (b)
50 mg of IR component is encapsulated with 50 mg of modified
release (MR) component to give a 20 mg dosage strength product.

| | % (w/w) |
|---|---|
| IR component | |
| Famciclovir | 20 |
| Microcrystalline cellulose | 50 |
| Lactose | 28 |
| Povidone | 2 |
| MR component | |
| Famciclovir | 20 |
| Microcrystalline cellulose | 50 |
| Eudragit ® RS | 28 |
| Povidone | 2 |

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A pharmaceutical composition comprising a first component of active ingredient-containing particles and at least one subsequent component of active ingredient-containing particles, wherein at least one of said components comprises famciclovir and at least one of said components further comprises a modified release coating, a modified release matrix material, or both, such that the composition, following oral delivery to a subject, delivers the active ingredient in a bimodal or multimodal manner.

2. The composition of claim 1 wherein each component comprises famciclovir-containing particles.

3. The composition of claim 1 wherein the composition comprises a first component of famciclovir-containing particles and one subsequent component of famciclovir-containing particles.

4. The composition of claim 3, wherein the first component comprises an immediate release component and the second component comprises a modified release component.

5. The composition of claim 1, wherein the active ingredient-containing particles are erodable.

6. The composition of claim 1, wherein at least one of said components further comprises a modified-release coating.

7. The composition of claim 1, wherein at least one of said components further comprises a modified-release matrix material.

8. The composition of claim 7, wherein said modified release matrix material is selected from the group consisting of hydrophilic polymers, hydrophobic polymers, natural polymers, synthetic polymers and mixtures thereof.

9. The composition of claim 8 wherein the famciclovir is released to the surrounding environment by erosion.

10. The composition of claim 9 wherein said composition further comprises an enhancer.

11. The composition of claim 8 comprising from about 0.1 mg to about 1 g of famciclovir.

12. A pharmaceutical composition comprising a first component of active ingredient-containing particles and at least one subsequent component of active ingredient-containing particles, wherein at least one of said components comprises famciclovir and at least one of said components further comprises a modified release coating, a modified release matrix material, or both, such that the composition, following oral delivery to a subject, delivers the active ingredient in a continuous manner.

13. The composition of claim 12 wherein each component comprises famciclovir-containing particles.

14. The composition of claim 12 wherein the composition comprises a first component of famciclovir-containing particles and one subsequent component of famciclovir-containing particles.

15. The composition of claim 14, wherein the first component comprises an immediate release component and the second component comprises a modified release component.

16. The composition of claim 12, wherein the active ingredient-containing particles are erodable.

17. The composition of claim 12, wherein at least one of said components further comprises a modified-release coating.

18. The composition of claim 12, wherein at least one of said components further comprises a modified-release matrix material.

19. The composition of claim 18, wherein said modified release matrix material is selected from the group consisting of hydrophilic polymers, hydrophobic polymers, natural polymers, synthetic polymers and mixtures thereof.

20. The composition of claim 19 wherein the famciclovir is released to the surrounding environment by erosion.

21. The composition of claim 20 wherein said composition further comprises an enhancer.

22. The composition of claim 19 comprising from about 0.1 mg to about 1 g of famciclovir.

23. A dosage form comprising the composition of claim 1.

24. The dosage form of claim 23 comprising a blend of active ingredient-containing particles contained within a hard gelatin or soft gelatin capsule.

25. The dosage form of claim 24, wherein the active ingredient-containing particles are in the form of mini-tablets and the capsule contains a mixture of said mini-tablets.

26. The dosage form of claim 25 in the form of tablet.

27. The dosage form of claim 26 wherein the famciclovir-containing particles are provided in a rapidly dissolving dosage form.

28. The dosage form of claim 26 wherein the tablet is a fast-melt tablet.

29. A dosage form comprising the composition of claim 12.

30. The dosage form of claim 29 comprising a blend of active ingredient-containing particles contained within a hard gelatin or soft gelatin capsule.

31. The dosage form of claim 30, wherein the active ingredient-containing particles are in the form of mini-tablets and the capsule contains a mixture of said mini-tablets.

32. The dosage form of claim 31 in the form of tablet.

33. The dosage form of claim 32 wherein the famciclovir-containing particles are provided in a rapidly dissolving dosage form.

34. The dosage form of claim 32 wherein the tablet is a fast-melt tablet.

35. A method for treating a viral infection comprising the step of administering a therapeutically effective amount of the composition of claim 1.

36. The method of claim 35, wherein said virus is selected from the group consisting of HSV-1, HSV-2, VZV, hepatitis B, and Epstein Barr.

37. A method for treating a viral infection comprising the step of administering a therapeutically effective amount of the composition of claim 12.

38. The method of claim 37, wherein said virus is selected from the group consisting of HSV-1, HSV-2 and VZV, hepatitis B, and Epstein Barr.

39. The composition of claim 1 wherein the modified-release coating comprises a pH-dependent polymer coating for releasing a pulse of the active ingredient in said patient following a time delay of about 6 to about 12 hours after administration of said composition to said patient.

40. The composition according to claim 39, wherein said polymer coating comprises methacrylate copolymers.

41. The composition according to claim 39, wherein the polymer coating comprises a mixture of methacrylate and ammoniomethacrylate copolymers in a ratio sufficient to achieve a pulse of the active ingredient following a time delay of at least about 6 hours.

42. The composition according to claim 41, wherein the ratio of methacrylate to ammonio methacrylate copolymers is approximately 1:1.
